(19) United States
(12) Patent Application Publication
(10) Pub. No.: US 2006/0127479 A1
(43) Pub. Date: Jun. 15, 2006

(54) Solvent free taste masked pharmaceutical compositions

(76) Inventors: Natrajan Kumaraperumal, Monmouth Junction, NJ (US); Suresh Palaniswamy, East Windsor, NJ (US); Pablo Davila, East Windsor, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/961,728

(22) Filed: Oct. 8, 2004

(51) Int. Cl. A61K 9/24

(52) U.S. Cl. 424/472

(57) ABSTRACT

A taste masked pharmaceutical composition comprising: (a) a core comprising a bitter tasting drug, such as cetirizine dihydrochloride; and (b) a coating comprising a pharmaceutically acceptable cationic co-polymer based on mono- or dialkylaminoalkyl methacrylate and neutral acrylic or methacrylic esters, wherein the alkyl group independently has 1 to 6 carbon atoms, wherein said coating is applied to the surface of said core. The taste masked pharmaceutical compositions of the invention may be prepared without using an organic solvent or a cyclodextrin.

### FIELD OF THE INVENTION

The present invention provides a taste masked pharmaceutical composition comprising (a) a core comprising a bitter tasting drug, such as cetirizine dihydrochloride; and (b) a coating comprising a cationic co-polymer based on mono- or dialkylaminoalkyl methacrylate and neutral acrylic or methacrylic esters, wherein said composition is prepared by a process which is essentially free of an organic solvent.

### BACKGROUND OF THE INVENTION

Cetirizine (C₂₁H₂₅ClN₂O₃) is also known as [2-[4-[(4-chlorophenyl)-phenylmethyl]-1-piperazinyl]ethoxy]acetic acid or [2-[4-[(p-chloro-α-phenylbenzyl)-1-piperazinyl]ethoxy]acetic acid. Cetirizine is a human metabolite of hydroxyzine. Cetirizine is useful as an antiallergen, spasmolytic, and a histamine H₂-antagonist and is generally non-sedating. See U.S. Pat. No. 4,525,358 (the '358 patent) and The Merck Index, Eleventh Edition, Page 310, Entry 2013 (1989). Cetirizine belongs to a class of drugs known as substituted benzhydrylpiperazines which characteristically have an extremely bitter taste.

Various techniques intended to mask the taste of such drugs have been described. Simple approaches include adding flavoring or sweetening ingredients to the compositions. When simple approaches are ineffective, various other approaches are used. An example of one such approach is to create a physical barrier to the drug from the saliva. Cationic co-polymers synthesized from dimethylaminoethylmethacrylate and neutral methacrylic acid have been employed as a barrier material. However, such barrier co-polymers generally require that an organic solvent be used during formulating. For example, U.S. Pat. No. 5,286,489 teaches ethyl alcohol as a solvent for a methyl methacrylic ester co-polymer. U.S. Pat. No. 4,708,867 teaches acetone and isopropyl alcohol as solvents for a first coating of polyvinylpyrrolidone, and a second coating of a dimethylaminoethyl and methyl methacrylate co-polymer. U.S. Pat. No. 4,760,093 teaches methylene chloride as a solvent for a dimethylaminoethyl and methyl methacrylate and neutral methacrylic acid esters.

U.S. Pat. No. 3,558,600 describes a method for masking the bitter taste of antihistaminic agents belonging to the substituted 1-(p-chloro-benzhydryl)piperazine family, which involves converting the active substance in free base form into the form of its salt with a long-chain alkyl sulphate, such as stearyl sulphate.

U.S. Pat. No. 4,525,358 describes cetirizine and methods for achieving an antiallergic, antihistaminic, bronchodilator and antispasmodic effect in a patient by administering cetirizine. This patent describes only one formulation which contains 100 mg of cetirizine, 67 mg of lactose, 1 mg of magnesium stearate and 2 mg of silicon dioxide.

U.S. Pat. No. 6,455,533 (the '533 patent) describes pharmaceutical compositions for oral administration containing an active substance belonging to the substituted benzhydrylpiperazine family and cyclodextrin. According to this patent there are two methods for masking the taste of the active substances in a solid pharmaceutical composition for oral administration. In the first method, an inclusion complex is formed between the active substance and a cyclodextrin. In the second method, an inclusion complex is not formed between the active substance and cyclodextrin. However in both methods, cyclodextrin must be present. The FDA's Orange Book lists the '533 patent as a formulation covering ZYRTEC® Chewable Tablets, which are commercially-available from Pfizer. The tablets contain 5 mg or 10 mg of cetirizine hydrochloride, acesulfame potassium, artificial grape flavour, betadex, NF (β-cyclodextrin), blue dye, colloidal silicon dioxide, lactose monohydrate, magnesium stearate, mannitol, microcrystalline cellulose, natural flavour and red dye (carmine).

U.S. Pat. No. 5,489,436 (the '436 patent) describes chewable tablets prepared from a coated medicament wherein the coating is a “reverse enteric coating” designed to be soluble at the acidic pH of the stomach but relatively insoluble in the mouth. The coatings comprise a polymer mixture of dimethylaminoethyl methacrylate and neutral methacrylic acid ester and a cellulose ester. The '436 patent does not teach substituted benzhydrylpiperazine compounds, and additionally requires a cellulose ester, such as cellulose acetate or cellulose triacetate, in the coating. In addition, the '436 patent teaches methanol and acetone as solvents for a polymer mixture of dimethylaminoethyl methacrylate and neutral methacrylic acid ester.

The prior art discussed above either requires the use of an organic solvent during the preparation of taste masked pharmaceutical compositions containing a cationic co-polymer of dimethylaminoethylmethacrylate and neutral methacrylic acid, or requires a cyclodextrin. However, organic solvents present handling issues in the workplace and are environmentally hazardous. Even residual organic solvents may deleteriously effect human health. Thus, it would be desirable to prepare a taste masked pharmaceutical composition for oral administration containing a cationic co-polymer of dimethylaminoethylmethacrylate and neutral methacrylic acid without the use of organic solvents.

### SUMMARY OF THE INVENTION

The invention provides a taste masked pharmaceutical composition comprising:
(a) a core comprising a bitter tasting drug, and optionally a binder and an inert carrier; and
(b) a coating comprising a pharmaceutically acceptable cationic co-polymer based on mono- or dialkylaminoalkyl methacrylate and neutral acrylic or methacrylic esters, wherein the alkyl group independently has 1 to 6 carbon atoms, wherein said coating is applied to the surface of said core, wherein said composition is prepared by a process which is essentially free of an organic solvent. The bitter tasting drug is preferably cetirizine dihydrochloride.

According to another aspect, the invention provides a process for preparing a taste masked pharmaceutical composition, said process comprising:
(i) preparing an aqueous solution or dispersion which comprises a bitter tasting drug and a binder;
(ii) applying the solution or dispersion formed in Step (i) onto an inert carrier to form core granules or agglomerates;
(iii) preparing a coating comprising an aqueous dispersion of a pharmaceutically acceptable cationic co-polymer based on mono- or dialkylaminoalkyl methacrylate and neutral acrylic or methacrylic esters, wherein the alkyl group independently has 1 to 6 carbon atoms; and
(iv) applying the coating formed in Step (iii) to the surface of the core granules or agglomerates formed in Step (ii) to form a composition, wherein the process is essentially free of an organic solvent.

According to another aspect, the invention provides a process for preparing a taste masked pharmaceutical composition, said process comprising: applying a coating on a bitter tasting drug, wherein the coating comprises an aqueous dispersion of a pharmaceutically acceptable cationic co-polymer based on mono- or di-alkylaminoalkyl methacrylate and neutral acrylic or methacrylic esters, wherein the alkyl group independently has 1 to 6 carbon atoms, to form a composition.

According to another aspect, the invention provides a process for preparing a taste masked pharmaceutical composition, said process comprising:
(I) preparing an aqueous solution or dispersion which comprises a bitter tasting drug and a binder;
(II) applying the solution or dispersion formed in Step (I) onto an inert carrier to form core granules or agglomerates;
(III) preparing a mixture comprising water and a surfactant;
(IV) adding the mixture prepared in Step (111) to an aqueous dispersion comprising a pharmaceutically acceptable cationic co-polymer based on mono- or di-alkylaminoalkyl methacrylate and neutral acrylic or methacrylic esters, wherein the alkyl group independently has 1 to 6 carbon atoms; and
(V) applying the dispersion formed in Step (IV) to the surface of the core granules or agglomerates formed in Step (II) to form a composition, wherein the process is essentially free of an organic solvent.

The present inventors have unexpectedly determined that the taste masked pharmaceutical compositions of the invention may be prepared without using an organic solvent or a cyclodextrin.

### DESCRIPTION OF THE INVENTION

The invention provides a taste masked pharmaceutical composition comprising:
(a) a core comprising a bitter tasting drug, and optionally a binder and an inert carrier; and
(b) a coating comprising a pharmaceutically acceptable cationic co-polymer based on mono- or dialkylaminoalkyl methacrylate and neutral acrylic or methacrylic esters, wherein the alkyl group independently has 1 to 6 carbon atoms, wherein said coating is applied to the surface of said core. The compositions of the invention are prepared by a process which is essentially free of an organic solvent. As used herein, “essentially free” means that an organic solvent is not used during the preparation of the composition of the invention. It is understood, however, that an organic solvent may be used to prepare one or more ingredients of the composition.

It is within the scope of the invention to coat the bitter tasting drug without first mixing the bitter tasting drug with a binder, inert carrier, or other excipients. For example, crystals of the bitter tasting drug may be coated with a pharmaceutically acceptable cationic co-polymer based on mono- or dialkylaminoalkyl methacrylate and neutral acrylic or methacrylic esters, wherein the alkyl group independently has 1 to 6 carbon atoms.

The bitter tasting drug is not limited with the proviso that it is used as a medically active component and has a bitter taste. Examples of such drugs include: central nervous system drugs such as a hypnotic sedative, a sleep inductor, an anxiolytic drug, an antiepileptic, an antipyretic-analgesic-anti-inflammatory drug, an antidepressant, a histamine H₂-antagonist, a 5-HT agonist, an antiparkinsonism drug and a psychoneurosis drug, circulatory drugs such as a skeletal muscle relaxant, an autonomic drug, an antispasmodic agent, a cardiotonic agent, an arrhythmia drug, a diuretic agent, an antihypertensive drug, a vasoconstrictor, a coronary vasodilator, a peripheral vasodilator and a hyperlipemia drug, allergy drugs such as an antitussive expectorant and a bronchodilator, digestive organ drugs such as an antidiarrheal drug, a drug for controlling intestinal function, an antiulcer drug, a stomatic digestive drug and an antacid agent and hormone drugs such as a pituitary hormone drug, a thyroid hormone drug and an anti-thyroid hormone drug, as well as a urogenital organ drug, a vitamin compound, a hemostatic drug, a blood coagulation inhibitor, a pulmonary disease drug, an antidote, a habitual intoxication drug, a gout treating drug, a diabetic drug, an anti-malignant tumor drug, an antibiotic, a chemotherapy drug, an anthelmintic drug and an anti-protozoan drug. The bitter tasting drug is preferably selected from a histamine H₂-antagonist, a 5-HT agonist, an antibiotic, or a nonsteriodal anti-inflammatory drug. More preferably, the bitter tasting drug is a histamine H₂-antagonist. A mixture of bitter tasting drugs may also be used.

Preferred bitter tasting drugs include, but are not limited to, loperamide, sildenafil, topiramate, citalopram, mirtazapine, desloratadine, enalapril, lorazepam, zopiclone, selegline, lorazepam, risperidone, ondansetron, olanzapine, almotriptan, frovatriptan, naratriptan, sumatriptan, zolmitriptan, rizatriptan, cimetidine, ranitidine, famotidine, nizatidine, etinidine, lupitidine, nifentidine, niperotidine, roxatidine, sulfotidine, tuvatidine, zaltidine, penicillin, ampicillin, erythromycin, acetaminophen, caffeine, dextromethorphan, diphenhydramine, theophylline, spironolactone, chloropheniramine, nabumetone, ibuprofen, naprosyn, ketoprofen, astemizole, azatadine, brompheniramine, cetirizine, chlorpheniramine, clemastine, cyproheptadine, dexchlorpheniramine, dimenhydrinate, diphenhydramine, doxylamine, hydroxyzine, loratadine, phenindamine, terfenadine, tripelennamine, effective salts thereof and derivatives thereof.

As used herein, “cetirizine” refers to [2-[4-[(4-chlorophenyl)-phenylmethyl]-1-piperazinyl]ethoxy]acetic acid or [2-[4-[(p-chloro-α-phenylbenzyl)-1-piperazinyl]ethoxy]acetic acid and pharmaceutically acceptable salts thereof. Preferred salts are acid addition salts, especially the dihydrochloride, referred to also herein as the “hydrochloride”. Most preferably, the bitter tasting drug is cetirizine dihydrochloride.

The bitter tasting drug is present in the pharmaceutical compositions in an amount of from about 0.1 weight percent (wt. %) to about 20 wt. %, based on the total weight of the pharmaceutical composition. Preferably, the bitter tasting drug is present in an amount of from about 1 wt. % to about 5 wt. %, more preferably about 2 wt. %, based on the total weight of the composition.

Examples of binders include, but are not limited to, methylcellulose, carboxymethycellulose sodium, ethyl cellulose, hydroxypropyl methylcellulose, hydroxyethyl cellulose, hydroxypropyl cellulose, polyvinyl pyrrolidone, gelatine, polyvinyl alcohol, acacia, tragacanth, guar, pectin, starch paste, pre-gelatinized starch, sucrose, corn syrup and sodium alginate. A preferred binder is polyvinylpyrrolidone. A mixture of binders may also be used.

The binder is present in the pharmaceutical compositions in an amount of from about 0.1 wt. % to about 20 wt. %, based on the total weight of the pharmaceutical composition. Preferably, the binder is present in an amount of from about 1 wt. % to about 5 wt. %, more preferably about 2 wt. %, based on the total weight of the composition.

The inert carrier may be water soluble or water insoluble. Examples of inert carriers include, but are not limited to, spray-dried or anhydrous lactose, sucrose, dextrose, starch, pre-gelatinized starch, mannitol, maltitol, sorbitol, xylitol, microcrystalline cellulose, dibasic calcium phosphate, tribasic calcium phosphate and calcium sulphate. A preferred inert carrier is microcrystalline cellulose. A mixture of inert carriers may also be used.

The inert carrier is present in the pharmaceutical compositions in an amount of from about 15 wt. % to about 80 wt. %, based on the total weight of the pharmaceutical composition. Preferably, the inert carrier is present in an amount of from about 40 wt. % to about 65 wt. %, more preferably about 54 wt. %, based on the total weight of the composition.

The coating component of the compositions of the invention contains a pharmaceutically acceptable cationic co-polymer based on mono- or di-alkylaminoalkyl methacrylate and neutral acrylic or methacrylic esters, wherein the alkyl group independently has 1 to 6 carbon atoms, preferably 1 to 4 carbon atoms. Preferably, the cationic co-polymer is based on dimethylaminoethyl methacrylate and neutral methacrylic acid esters (EUDRAGIT® E 100). EUDRAGIT® E 100 is available from Degussa Rohm Pharma Polymers. EUDRAGIT® EPO is a powdered form of EUDRAGIT® E 100. In addition, EUDRAGIT® E 100 is also known as aminoalkylalkyl methacrylate copolymer E and basic butylated methacrylate copolymer E.

The cationic co-polymer based on mono- or di-alkylaminoalkyl methacrylate and neutral acrylic or methacrylic esters is preferably soluble in acidic environments where the pH is up to about 5. At a pH greater than about 5, the co-polymer is preferably insoluble in water. Thus, the co-polymer is preferably insoluble in the mouth of a patient where the pH is about 7.

The cationic co-polymer is present in an amount of from about 0.5 wt. % to about 15 wt. %, based on the total weight of the pharmaceutical composition. Preferably, the cationic co-polymer is present in an amount of from about 1 wt. % to about 5 wt. %, more preferably about 3 wt. %, based on the total weight of the composition.

According to the process of the invention, the coating is applied to the granules in an amount which provides a taste masking effect for a relatively short period during which the composition, for example, is chewed by the patient, and which allows the dosage form to be broken into smaller particles allowing it to be easily swallowed, making it more palatable which results in better patient compliance.

The pharmaceutical compositions of the invention may contain one or more excipients in addition to the binder and inert carrier previously mentioned to enhance the palatability of the dosage form and to improve the taste. Examples of excipients include, but are not limited to, diluents, fillers/bulking agents, effervesant salts, disintegrants, lubricants, glidants, emulsifiers, electrolytes, wetting agents, solubilizers, surfactants, colors, flavors, pigments, anti-caking agents, sweeteners, and effervescent couples. A mixture of excipients may also be used. Such excipients are known to those skilled in the art, and thus, only a limited number will be specifically referenced. Preferably, the excipients meet the standards of the National Formulary (NF) or United States Pharmacopoeia (USP).

Examples of disintegrants include:
(i) cross-linked polyvinylpyrrolidones, e.g., crospovidones, such as Polyplasdone® XL and Kollidon® CL;
(ii) alginic acid and sodium alginate;
(iii) methacrylic acid-divinylbenzene co-polymer salts, e.g., Amberlite® IRP-88; and
(iv) cross-linked sodium carboxymethylcellulose, available as, e.g., Ac-di-sol®, Primellose®, Pharmacel® XL, Explocel® and Nymcel® ZSX.
   Additional disintegrants also include hydroxypropylmethyl cellulose, croscarmellose sodium, polacrillin potassium, polyacrylates, such as Carbopol®, magnesium aluminium silicate and bentonite.

Examples of glidants include, but are not limited to, silica, magnesium trisilicate, powdered cellulose, starch, talc and tribasic calcium phosphate. A preferred glidant is talc.

Examples of lubricants include, but are not limited to, vegetable oils, such as hydrogenated vegetable oil or hydrogenated castor oil; polyethylene glycols, such as polyethylene glycol (PEG)-4000 and PEG-6000; stearic acid; salts of stearic acid, such as magnesium stearate, sodium stearate and sodium stearyl fumarate; mineral salts, such as talc; inorganic salts; organic salts, such as sodium benzoate, sodium acetate and sodium oleate; and polyvinyl alcohols.

Examples of preferred surfactants include:
1) reaction products of a natural or hydrogenated castor oil and ethylene oxide. The natural or hydrogenated castor oil may be reacted with ethylene oxide in a molar ratio of from about 1:35 to about 1:60, with optional removal of the PEG component from the products. Various such surfactants are commercially-available. The PEG-hydrogenated castor oils, available under the trademark CREMOPHOR, are especially suitable. Particularly suitable are CREMOPHOR RH 40, which has a saponification number of about 50 to 60, an acid number less than about 1, a water content (Fischer) less than about 2%, an n_{D}⁶⁰ of about 1.453 to 1.457 and an HLB of about 14 to 16; and CREMOPHOR RH 60, which has a saponification number of about 40 to 50, an acid number less than about 1, an iodine number of less than about 1, a water content (Fischer) of about 4.5 to 5.5%, an n_{D}²⁵ of about 1.453 to 1.457 and an HLB of about 15 to 17. An especially preferred product of this class is CREMOPHOR RH 40. Also suitable are PEG castor oils, such as that available under the trade name CREMOPHOR EL, which has a molecular weight (by steam osmometry) of about 1630, a saponification number of about 65 to 70, an acid number of about 2, an iodine number of about 28 to 32 and an n_{D}²⁵ of about 1.471.
2) Polyoxyethylene-sorbitan-fatty acid esters, also called polysorbates, e.g., mono- and tri-lauryl, palmityl, stearyl and oleyl esters of the type known and commercially-available under the trademark TWEEN (Fiedler, loc. cit., p. 1300-1304) including the products TWEEN:
   20 [polyoxyethylene(20)sorbitanmonolaurate],
   21 [polyoxyethylene(4)sorbitanmonolaurate],
   40 [polyoxyethylene(20)sorbitanmonopalmitate],
   60 [polyoxyethylene(20)sorbitanmonostearate],
   65 [polyoxyethylene(20)sorbitantristearate],
   80 [polyoxyethylene(20)sorbitanmonooleate],
   81 [polyoxyethylene(5)sorbitanmonooleate] and
   85 [polyoxyethylene(20)sorbitantrioleate].
Especially preferred products of this class are TWEEN 40 and TWEEN 80.
Although PEG itself does not function as a surfactant, a variety of PEG-fatty acid esters have useful surfactant properties. Among the PEG-fatty acid monoesters, esters of lauric acid, oleic acid and stearic acid are most useful.
3) Polyoxyethylene fatty acid esters, e.g., polyoxyethylene stearic acid esters of the type known and commercially-available under the trademark MYRJ (Fiedler, loc. cit., 2, pp. 834-835). An especially preferred product of this class is MYRJ 52 having a D25 of about 1.1., a melting point of about 40 to 44° C., an HLB value of about 16.9, an acid value of about 0 to 1 and a saponification no. of about 25 to 35.
4) Polyoxyethylene-polyoxypropylene co-polymers and block co-polymers, e.g., of the type known and commercially-available under the trademark PLURONIC, EMKALYX and POLOXAMER (Fiedler, loc. cit., 2, p. 959). An especially preferred product of this class is PLURONIC F68, having a melting point of about 52° C. and a molecular weight of about 6800 to 8975. A further preferred product of this class is POLOXAMER 188.
5) Dioctylsulfosuccinate or di-[2-ethylhexyl]-succinate (Fiedler, loc. cit., 1, p. 107-108).
6) Phospholipids, in particular, lecithins (Fiedler, loc. cit., 2, pp. 943-944). Suitable lecithins include, in particular, soybean lecithins.
7) PEG mono- and di-fatty acid esters, such as PEG dicaprylate, also known and commercially-available under the trademark MIGLYOL 840, PEG dilaurate, PEG hydroxystearate, PEG isostearate, PEG laurate, PEG ricinoleate, PEG stearate and so forth (Fiedler, loc. cit., 2, pp. 808-809).
8) Polyoxyethylene alkyl ethers, such as those commercially-available under the trademark BRIJ, e.g., Brij 92V and Brij 35.
9) Fatty Acid Monoglycerides, e.g., glycerol monostearate and glycerol monolaurate.
10) Tocopherol esters, e.g., tocopheryl acetate and tocopheryl acid succinate.
11) Docusate salts, e.g., dioctylsulfosuccinate or related compounds, such as di-[2-ethylhexyl]-succinate (Fiedler, loc. cit., 1, pp. 107-108).

More preferably, the surfactant is selected from polyoxyethylene(20)sorbitanmonooleate, glycerol monostearate, glycerol monolaurate, glycerol monopalmitate, glycerol monooleate, glycerol monocaprylate, sodium lauryl sulphate, cetyltrimethyl ammoniumbromide, and dioctylsodium sulfosuccinate. A mixture of surfactants may also be used.

In one embodiment of the invention, the pharmaceutical composition is in the form of a solid dosage form which contains coated granules comprising a bitter tasting drug, a binder, an inert carrier, a cationic co-polymer based on mono- or di-alkylaminoalkyl methacrylate and neutral acrylic or methacrylic esters, and a mixture of excipients. A preferred ratio of the mixture of excipients, including the amount of binder and diluent, to the coated granules is from about 0.4:1 to about 5:1. More preferably, the ratio is from about 0.4:1 to about 2:1. Most preferably, the ratio is about 0.6:1.

One of the advantages of the pharmaceutical compositions of the invention is that they provide a method for formulating extremely palatable solid dosage forms which contain bitter tasting drugs. Solid dosage forms include sprinkles, capsules, caplets, powders and tablets. In one embodiment, the compositions are compressed into a tablet. Tablets may include multiple layer compressed tablets, bi-layer tablets, effervescent tablets, mouth dissolving tablets, water dispersible tablets, and chewable tablets. Preferably, the tablets are chewable, orally disintegrating or dissolving. The tablet formulation can be prepared by wet granulation, dry granulation, direct compression or by any other technique known in the pharmaceutical art.

In a preferred embodiment of the invention, the pharmaceutical composition is in the form of a chewable tablet which comprises:
(a) a core comprising cetirizine dihydrochloride, polyvinyl pyrrolidone and microcrystalline cellulose; and
(b) a coating comprising a cationic co-polymer of dimethylaminoethyl methacrylate and neutral methacrylic acid esters, wherein said coating is applied to the surface of the core, wherein said composition is prepared by a process which is essentially free of an organic solvent.

In one embodiment of the invention, the taste masked pharmaceutical compositions are prepared by a process comprising:
(i) preparing an aqueous solution which comprises a bitter tasting drug and a binder;
(ii) applying the solution formed in Step (i) onto an inert carrier to form core granules or agglomerates;
(iii) preparing a coating comprising an aqueous dispersion of a pharmaceutically acceptable cationic co-polymer based on mono- or di-alkylaminoalkyl methacrylate and neutral acrylic or methacrylic esters, wherein the alkyl group independently has 1 to 6 carbon atoms;
(iv) applying the coating formed in Step (iii) to the surface of the core granules or agglomerates formed in Step (ii) to form a pharmaceutical composition; and
(v) compressing the composition formed in Step (iv) to form a tablet.

In one embodiment of the invention, the taste masked pharmaceutical compositions are prepared by a process comprising: applying a coating on a bitter tasting drug, wherein the coating comprises an aqueous dispersion of a pharmaceutically acceptable cationic co-polymer based on mono- or di-alkylaminoalkyl methacrylate and neutral acrylic or methacrylic esters, wherein the alkyl group independently has 1 to 6 carbon atoms, to form a pharmaceutical composition.

In one embodiment of the invention, the taste masked pharmaceutical compositions are prepared by a process comprising:
(I) preparing an aqueous solution or dispersion which comprises a bitter tasting drug and a binder;
(II) applying the solution or dispersion formed in Step (I) onto an inert carrier to form core granules or agglomerates;
(III) preparing a mixture comprising water and a surfactant, preferably, the surfactant is selected from glycerol monostearate, glycerol monolaurate, glycerol monopalmitate, glycerol monooleate, and glycerol monocaprylate;
(IV) adding the mixture prepared in Step (III) to an aqueous dispersion comprising a pharmaceutically acceptable cationic co-polymer based on mono- or di-alkylaminoalkyl methacrylate and neutral acrylic or methacrylic esters, and optionally a surfactant which preferably is sodium lauryl sulphate, wherein the alkyl group independently has 1 to 6 carbon atoms; and
(V) applying the dispersion formed in Step (IV) to the surface of the core granules or agglomerates formed in Step (II) to form a pharmaceutical composition; and
(VI) compressing the composition formed in Step (V) to form a tablet.

According to the processes of the invention, the loading of the bitter drug as a solution or dispersion over the core granules or agglomerates may be carried out by one or more granulation, spray coating, or coacervation techniques. The core granules or agglomerates prepared in the processes according to the invention, preferably have a particle size from about 180 to 420 microns.

The following non-limiting examples illustrate further aspects of the invention.

### EXAMPLE 1

Preparation of a Chewable Tablet Composition Containing 5 mg of Cetirizine Dihydrochloride.

| | % Based on Total |
|---|---|
| | Tablet Weight |
| Ingredient | (% w/w) |
| Cetirizine Hydrochloride | 2 |
| Polyvinylpyrrolidone (Povidone K 30) | 2 |
| Microcrystalline Cellulose | 54 |
| (Avicel ® PH 200) | |
| Purified Water | q.s. for 30% w/v |
| EUDRAGIT ® EPO* | 2.9 |
| Sodium Lauryl Sulfate | 0.29 |
| Talc | 2.2 |
| Purified Water | q.s. for 12.8% w/v |
| Mannitol | 21.64 |
| Xylitol | 8.0 |
| Colloidal Silicon Dioxide | 0.25 |
| Acesulfame Potassium | 0.88 |
| Microcrystalline Cellulose | 4.0 |
| and Guar Gum (Avicel ® CE15) | |
| Flavors | 0.6 |
| Dyes | 0.24 |
| Magnesium Stearate | 1.0 |

*EUDRAGIT ® EPO is the powder form of EUDRAGIT ® E100.

The tablets are prepared according to the following procedure:
1. Dissolve cetirizine hydrochloride in purified water and add Povidone K 30 to form a solution.
2. Spray the solution prepared in Step 1 onto Avicel® PH 200 granules in a fluidized bed apparatus with top spray setting under optimal processing conditions to yield core granules.
3. In a beaker, dissolve sodium lauryl sulfate in 66.67% of the total quantity of purified water for the coating step.
4. To Step 3, disperse EUDRAGIT® EPO and stir under low shear conditions and mix until an uniform dispersion is obtained.
5. In a separate beaker prepare a dispersion of talc in the remaining quantity of purified water.
6. Add the talc dispersion to Step 4 with stirring.
7. Spray the taste masking dispersion onto the core granules of Step 2 to obtain taste masked granules.
8. Mix the granules prepared in Step 7 with the following excipients which were passed through a #30 mesh screen: mannitol, xylitol, colloidal silicone dioxide, acesulfame potassium, Avicel® CE 15, flavors, dyes, and magnesium stearate.
9. Compress the granules prepared in Step 8 using a Korsch XL100 Automatic Tablet Press into tablets.

### EXAMPLE 2

Preparation of a Chewable Tablet Composition Containing 5 mg of Cetirizine Dihydrochloride.

| | % Based on Total |
|---|---|
| | Tablet Weight |
| Ingredient | (% w/w) |
| Cetirizine Hydrochloride | 2 |
| Polyvinylpyrrolidone (Povidone K 30) | 2 |
| Microcrystalline Cellulose | 54 |
| (Avicel ® PH 200) | |
| Purified Water | q.s. for 30% w/v |
| EUDRAGIT ® EPO | 2.9 |
| Glycerol monostearate | 0.48 |
| Sodium Lauryl Sulfate | 0.29 |
| Polysorbate 80 | 0.07 |
| Talc | 1.25 |
| Purified Water | q.s. for 15.6% w/v |
| Mannitol | 21.64 |
| Xylitol | 8.4 |
| Colloidal Silicon Dioxide | 0.25 |
| Acesulfame Potassium | 0.88 |
| Microcrystalline Cellulose | 4.0 |
| and Guar Gum (Avicel ® CE15) | |
| Flavors | 0.6 |
| Dyes | 0.24 |
| Magnesium Stearate | 1.0 |

*EUDRAGIT ® EPO is the powder form of EUDRAGIT ® E100.

The tablets are prepared according to the following procedure:
1. Dissolve cetirizine hydrochloride in purified water and add Povidone K 30 to form a solution.
2. Spray the solution prepared in Step 1 onto Avicel PH 200 granules in a fluidized bed apparatus with top spray setting under optimal processing conditions to yield core granules.
3. In a beaker, dissolve sodium lauryl sulfate in 41.67% of the total quantity of purified water for the coating step.
4. To Step 3, disperse EUDRAGIT® EPO and stir under low shear conditions and mix until an uniform dispersion is obtained.
5. Prepare an oil-in-water emulsion of glycerol monostearate in purified water (using 16.67% of the total quantity of purified water for the coating step) and polysorbate 80 as surfactant.
6. Add the emulsion of Step 5 to the dispersion of Step 4 slowly.
7. In a separate beaker prepare a dispersion of talc in the remaining quantity of purified water.
8. Add the talc dispersion to Step 7 under stirring.
9. Spray the taste masking dispersion onto the core granules of Step 2 to obtain taste masked granules.
10. Mix the granules prepared in Step 9 with the following excipients which were passed through a #30 mesh screen: mannitol, xylitol, colloidal silicone dioxide, acesulfame potassium, Avicel® CE 15, flavors, dyes, and magnesium stearate.
11. Compress the granules prepared in Step 10 using a Korsch XL100 Automatic Tablet Press into tablets.

While the invention has been described with particular reference to certain embodiments thereof, it will be understood that changes and modifications may be made by those of ordinary skill within the scope and spirit of the following claims:

What is claimed is:

1. A taste masked pharmaceutical composition comprising:
(a) a core comprising a bitter tasting drug; and
(b) a coating comprising a pharmaceutically acceptable cationic co-polymer based on mono- or dialkylaminoalkyl methacrylate and neutral acrylic or methacrylic esters, wherein the alkyl group independently has 1 to 6 carbon atoms, wherein said coating is applied to the surface of said core, wherein said composition is prepared by a process which is essentially free of an organic solvent.

2. A taste masked pharmaceutical composition comprising:
(a) a core comprising a bitter tasting drug, a binder; and an inert carrier, wherein the bitter tasting drug is selected from the group consisting of a central nervous system drug, a sleep inductor, an anxiolytic drug, an antiepileptic, an antipyretic-analgesic-anti-inflammatory drug, an antidepressant, a histamine H₂-antagonist, a 5-HT agonist, an antiparkinsonism drug, a psychoneurosis drug, a skeletal muscle relaxant, an autonomic drug, an antispasmodic agent, a cardiotonic agent, an arrhythmia drug, a diuretic agent, an antihypertensive drug, a vasoconstrictor, a coronary vasodilator, a peripheral vasodilator and a hyperlipemia drug, an antitussive expectorant, a bronchodilator, an antidiarrheal drug, a drug for controlling intestinal function, an antiulcer drug, a stomatic digestive drug, an antacid, a pituitary hormone drug, a thyroid hormone drug, an anti-thyroid hormone drug, a urogenital organ drug, a vitamin compound, a hemostatic drug, a blood coagulation inhibitor, a pulmonary disease drug, an antidote, a habitual intoxication drug, a gout treating drug, a diabetic drug, an anti-malignant tumor drug, an antibiotic, a chemotherapy drug, an anthelmintic drug, an anti-protozoan drug, and mixtures thereof; and
(b) a coating comprising a pharmaceutically acceptable cationic co-polymer based on mono- or dialkylaminoalkyl methacrylate and neutral acrylic or methacrylic esters, wherein the alkyl group independently has 1 to 6 carbon atoms, wherein said coating is applied to the surface of said core, wherein said composition is prepared by a process which is essentially free of an organic solvent.

3. The composition according to claim 2, wherein the bitter tasting drug is selected from the group consisting of a histamine H₂-antagonist, a 5-HT agonist, an antibiotic, and a nonsteriodal anti-inflammatory drug.

4. The composition according to claim 3, wherein the bitter tasting drug is a histamine H₂-antagonist.

5. The composition according to claim 1, wherein the bitter tasting drug is selected from the group consisting of loperamide, sildenafil, topiramate, citalopram, mirtazapine, desloratadine, enalapril, lorazepam, zopiclone, selegline, lorazepam, risperidone, ondansetron, olanzapine, almotriptan, frovatriptan, naratriptan, sumatriptan, zolmitriptan, rizatriptan, cimetidine, ranitidine, famotidine, nizatidine, etinidine, lupitidine, nifentidine, niperotidine, roxatidine, sulfotidine, tuvatidine, zaltidine, penicillin, ampicillin, erythromycin, acetaminophen, caffeine, dextromethorphan, diphenhydramine, theophylline, spironolactone, chloropheniramine, nabumetone, ibuprofen, naprosyn, ketoprofen, astemizole, azatadine, brompheniramine, cetirizine, chlorpheniramine, clemastine, cyproheptadine, dexchlorpheniramine, dimenhydrinate, diphenhydramine, doxylamine, hydroxyzine, loratadine, phenindamine, terfenadine, tripelennamine, effective salts thereof, derivatives thereof, and mixtures thereof.

6. A taste masked pharmaceutical composition in the form of a solid dosage form which comprises:
(a) a core comprising cetirizine or a pharmaceutically acceptable salt thereof, polyvinyl pyrrolidone and microcrystalline cellulose; and
(b) a coating comprising a cationic co-polymer of dimethylaminoethyl methacrylate and neutral methacrylic acid esters, wherein said coating is applied to the surface of the core, wherein said composition is prepared by a process which is essentially free of an organic solvent.

7. The composition according to claim 6, wherein the cetirizine salt is cetirizine dihydrochloride.

8. The composition according to claim 6, wherein the solid dosage form is selected from the group consisting of sprinkles, a capsule, a caplet, a powder, multiple layer tablet, bi-layer tablet, effervescent tablet, mouth dissolving tablet, water dispersible tablet, and chewable tablet.

9. The composition according to claim 2, wherein the binder is selected from the group consisting of methylcellulose, carboxymethycellulose sodium, ethyl cellulose, hydroxypropyl methylcellulose, hydroxyethyl cellulose, hydroxypropyl cellulose; polyvinyl pyrrolidone, gelatine, polyvinyl alcohol, acacia, tragacanth, guar gum, pectin, starch paste, pre-gelatinized starch, sucrose, corn syrup, sodium alginate and mixtures thereof.

10. The composition according to claim 9, wherein the binder is polyvinylpyrrolidone.

11. The composition according to claim 2, wherein the inert carrier is selected from the group consisting of spray-dried or anhydrous lactose, sucrose, dextrose, starch, pre-gelatinized starch, mannitol, maltitol, sorbitol, xylitol, microcrystalline cellulose, dibasic calcium phosphate, tribasic calcium phosphate, calcium sulphate and mixtures thereof.

12. The composition according to claim 11, wherein the inert carrier is microcrystalline cellulose.

13. A process for preparing a taste masked pharmaceutical composition, said process comprising:
(i) preparing an aqueous solution or dispersion which comprises a bitter tasting drug and a binder;
(ii) applying the solution or dispersion formed in Step (i) onto an inert carrier to form core granules or agglomerates;
(iii) preparing a coating comprising an aqueous dispersion of a pharmaceutically acceptable cationic co-polymer based on mono- or dialkylaminoalkyl methacrylate and neutral acrylic or methacrylic esters, wherein the alkyl group independently has 1 to 6 carbon atoms; and
(iv) applying the coating formed in Step (iii) to the surface of the core granules or agglomerates formed in Step (ii) to form a composition, wherein the process is essentially free of an organic solvent.

14. The process according to claim 13, wherein the coating additionally comprises a surfactant.

15. The process according to claim 14, wherein the surfactant is selected from the group consisting of reaction products of a natural or hydrogenated castor oil and ethylene oxide, polyoxyethylene-sorbitan-fatty acid esters, polyoxyethylene fatty acid esters, polyoxyethylene-polyoxypropylene co-polymers and block co-polymers, dioctylsulfosuccinate or di-[2-ethylhexyl]-succinate, phospholipids, propylene glycol mono- and di-fatty acid esters, polyoxyethylene alkyl ethers, fatty acid monoglycerides, tocopherol esters, docusate salts and mixtures thereof.

16. The process according to claim 15, wherein the surfactant is selected from the group consisting of polyoxyethylene(20)sorbitanmonooleate, glycerol monostearate, glycerol monolaurate, glycerol monopalmitate, glycerol monooleate, glycerol monocaprylate, sodium lauryl sulphate, cetyltrimethyl ammoniumbromide, and dioctylsodium sulfosuccinate.

17. The process according to claim 13, which additionally comprises Step (v) compressing the composition formed in Step (iv) to form a tablet.

18. A process for preparing a taste masked pharmaceutical composition, said process comprising:
(I) preparing an aqueous solution or dispersion which comprises a bitter tasting drug and a binder;
(II) applying the solution or dispersion formed in Step (I) onto an inert carrier to form core granules or agglomerates;
(III) preparing a mixture comprising water and a surfactant;
(IV) adding the mixture prepared in Step (III) to an aqueous dispersion comprising a pharmaceutically acceptable cationic co-polymer based on mono- or di-alkylaminoalkyl methacrylate and neutral acrylic or methacrylic esters, wherein the alkyl group independently has 1 to 6 carbon atoms; and
(V) applying the dispersion formed in Step (IV) to the surface of the core granules or agglomerates formed in Step (II) to form a composition, wherein the process is essentially free of an organic solvent.

19. The process according to claim 18, wherein the surfactant in Step (III) is selected from the group consisting of glycerol monostearate, glycerol monolaurate, glycerol monopalmitate, glycerol monooleate, and glycerol monocaprylate.

20. The process according to claim 19, wherein the surfactant in Step (III) is glycerol monostearate.

21. The process according to claim 18, wherein the dispersion in Step (IV) additionally comprises a surfactant prior to addition of the mixture.

22. The process according to claim 21, wherein the surfactant is sodium lauryl sulfate.

23. The process according to claim 18, which additionally comprises Step (VI) compressing the composition formed in Step (V) to form a tablet.

24. A process for preparing a taste masked pharmaceutical composition, said process comprising: applying a coating on a bitter tasting drug, wherein the coating comprises an aqueous dispersion of a pharmaceutically acceptable cationic co-polymer based on mono- or di-alkylaminoalkyl methacrylate and neutral acrylic or methacrylic esters, wherein the alkyl group independently has 1 to 6 carbon atoms, to form a composition.

25. The composition according to claim 1, wherein the bitter tasting drug is present in an amount of from about 0.1 weight percent to about 20 weight percent, based on the total weight of the composition.

26. The composition according to claim 25, wherein the bitter tasting drug is present in an amount of from about 1 weight percent to about 5 weight percent, based on the total weight of the composition.

27. The composition according to claim 2, wherein the amount of the binder is from about 0.1 weight percent to about 20 weight percent, based on the total weight of the composition.

28. The composition according to claim 27, wherein the amount of the binder is from about 1 weight percent to about 5 weight percent, based on the total weight of the composition.

29. The composition according to claim 2, wherein the amount of the inert carrier is from about 15 weight percent to about 80 weight percent, based on the total weight of the composition.

30. The composition according to claim 29, wherein the amount of the inert carrier is from about 40 weight percent to about 65 weight percent, based on the total weight of the composition.

31. The composition according to claim 1, wherein the amount of the cationic co-polymer based on mono- or di-alkylaminoalkyl methacrylate and neutral acrylic or methacrylic esters is from about 0.5 weight percent to about 15 weight percent, based on the total weight of the composition.

32. The composition according to claim 31, wherein the amount of the cationic co-polymer is from about 1 weight percent to about 5 weight percent, based on the total weight of the composition.

33. The composition according to claim 2, which additionally comprises one or more excipients in addition to the binder and inert carrier.

34. A method of treating a disease or disorder in a subject in need thereof comprising administering to the subject a therapeutically effective amount of the pharmaceutical composition according to claim 1.
